(19) United States
(12) Patent Application Publication
(10) Pub. No.: US 2004/0029751 A1
(43) Pub. Date: Feb. 12, 2004

(54) Preparatiom for douches and/or irrigations of natural or pathological cavities in the human body

(76) Inventor: Beniamino Palmieri, Modena (IT)

Correspondence Address: Walter H Schneider, 21530 Beechwood Road, Circleville, OH, 43113, US

(21) Appl. No.: 10/450,398

(22) PCT Filed: Dec. 20, 2001
(86) PCT No.: PCT/EP01/15114

(30) Foreign Application Priority Data
Jan. 4, 2001 (IT) M02001A000002

(51) Int. Cl. A61K 7/40

(52) U.S. Cl. 510/131

(57) ABSTRACT

A preparation for douches and/or irrigations of natural or pathological cavities in the human body which comprises water added with at least one gaseous substance selected from carbon dioxide, oxygen, nitrogen, gaseous anaesthetic substances and inert gases.

The present invention relates to a preparation for douches and/or irrigations of natural or pathological cavities in the human body, and is designed for pre-, intra- and/or post-operative use, for example for hydrodissection, debridement and irrigation of arteriotomies, brain irrigations, irrigations of bloody parenchymal surfaces, etc.

In accordance with the prior art, preparations are known which are designed for douches and/or irrigations of natural or pathological cavities in the human body and contain active constituents suitable for debridement, deodorisation, disinfection, desensitisation, etc., in the form of liquids, foams, gels, etc.

The use of these active constituents always involves the risk of allergic reactions or local hypersensitivity in the patient. In addition, said preparations are not always effective when it is necessary to remove bacterial agglomerates, filaments of mucous or mucopus, etc., from said cavities.

The purpose of the present invention is to provide a preparation for douches and/or irrigations of natural or pathological cavities in the human body which has an effective cleansing and bacteriostatic action and does not trigger, or at least minimises the risk of allergic reactions or local hypersensitivity.

The present invention relates to a preparation for douches and/or irrigations of natural or pathological cavities in the human body, characterised in that it comprises water to which gaseous substances are added, said gaseous substances including, individually or in combination with one another, carbon dioxide, oxygen, nitrogen, gaseous anaesthetic substances and inert gases.

Carbon dioxide allows the pH of the preparation to be adjusted in a simple, controllable way to adapt it to the particular type of natural or pathological cavity to be cleansed or irrigated, because the pH of the preparation varies when the quantity of carbon dioxide dissolved in the water is varied.

Oxygen, added in a quantity which does not cause oxidative phenomena or tissue damages, optimises the therapeutic effects of the preparation of the invention.

Moreover, carbon dioxide, which develops in the form of bubbles when the preparation is introduced into said cavities, performs a bacteriostatic action, in particular against microaerophilic and aerobic germs.

The development of said gaseous substances in the form of bubbles gives rise to a mechanical action which facilitates the removal of bacterial agglomerates, filaments of mucous, mucopus etc. from the interior of said cavities.

Furthermore, carbon dioxide stimulates hyperaemia of the surface capillaries of the walls of said cavities, activating the trophism of the mucosal walls, and facilitates the normalisation of antibody responses.

In particular, in irrigations performed during laparoscopic surgery, when carbon dioxide is insufflated into the patient's abdomen, the use of the preparation of the invention enables the required carbon dioxide concentration to be maintained in the patient's abdomen during the irrigation stage. Moreover, carbon dioxide can be bubbled, together with other active constituents, over the entire peritoneal surface in order to facilitate the diffusion of said active constituents and obtain particular pharmacological effects, such as macrophagic activation of the mesothelial cells, stimulation of fibrinolytic effects to prevent the formation of adhesions, etc.

The preparation of the invention may include mineral water with a variable concentration of carbon dioxide, preferably ranging between approximately 1% and approximately 30%, with a preferred concentration of approximately 10%, and/or an oxygen concentration of up to 300-600 mg/litre.

The carbon dioxide can be added to the water by industrial processes of known type or can be performed in an extempore manner, for example by mixing citric acid and potassium bicarbonate in equal parts, diluted in water and lactic acid.

Oxygen can be added by bubbling under pressure, sonication, or other known chemical or physical methods of dissolving oxygen in water.

The preparation may also include substances with a cleansing, deodorising, desensitising, anti-itching, anti-inflammatory, wound-healing, revulsive, eudermic and reparatory, antibacterial, antiparasitic, antifingal, antiviral, antiexudative, corticosteroid, antitumoral, immunomodulating, haemostatic, haemorheological and hormonal activity.

Said substances should preferably have a formulation which does not form precipitates or insoluble salts on reacting with the carbon dioxide dissolved in the water, thus preventing its development in the form of bubbles. The use of substances that form precipitates or insoluble salts on reacting with carbon dioxide is acceptable if their concentration or formulation does not significantly reduce the development of carbon dioxide in bubble form during the use of the preparation of the invention.

The preparation of the invention could also contain bacteria, such as lactobacilli or other carbon-dioxide-producing bacteria, which are able to live in an acid environment and restore the bacterial flora in natural body cavities suffering from damage caused by dysmicrobism, such as the vagina, colorectum, urethra, bladder, etc.

The preparation of the invention can be advantageously used to aid penetration of genetic material into the cells, such as carriers or viruses, thus facilitating the transmembranal passage of the material thanks to the carbon dioxide dissolved in the water, which aids the passage of said genetic material through the cell membranes. Similarly, the action of carbon dioxide facilitates the absorption by the cells of antitumoral chemotherapy substances, thus making their action faster and more effective.

The use of the preparation of the invention is particularly advantageous for pre-, intra, and post-operative irrigations of the bladder cavity and/or the prostatic bed and/or the renal pelvis after prostatectomy, in view of its ability to aid the removal of fibrin fragments, inflammatory cells, mucous etc. as a result of the mechanical action exerted by the development of carbon dioxide bubbles. The use of the preparation of the invention for irrigations of the renal pelvis and urethral duct is also particularly advantageous for patients with indwelling catheters and for the hygiene of the urethral meatus, the male external genitals and the female urethra, especially in cases of chronic bacterial, fungal or vital infections.

In the female genital apparatus, the use of the preparation of the invention to irrigate the clitoris can accentuate the sensitivity and congestion of that anatomical structure, thus curing symptoms leading to frigidity and dyspareunia. The use of the preparation of the invention for bladder irrigations is particularly advantageous in case of cystitis with frequent, urgent urination, because the stimulation of hyperaemia of the capillaries on the bladder surface performed by carbon dioxide aids the elimination of toxic waste and the penetration into that wall of long-acting desensitising or anaesthetising active constituents which can be added to the preparation of the invention.

The hyperaemic action of carbon dioxide is advantageous in all cases requiring penetration of active constituents such as intravescical instillations of chemotherapeutics or BCG (tuberculin bacillus extract).

The hyperaemisation effect can be regulated by heating or cooling the preparation of the invention before introducing it into the cavity to be washed or irrigated.

The preparation of the invention can also be used for aerosol, for lavages after disobstruction of the auditory duct, the fallopian tubes, the spermatic ducts in general, for lavages after surgical disobstruction or for dilating arterias and facilitate their debridment.

More particularly, when the preparation of the invention contains large amounts of dissolved carbon dioxide, can include active constituents to make a parenteral perfusion solution, which can also be used as a dialysis solution for the peritoneal, pleural, pericardial or cranial joint cavity, to aid the removal of toxic solutes. The preparation of the invention can also advantageously include formed elements of blood or plasma for use as transfusion preparations with an antibacterial or haemodilution function. In this case, it is advantageous to include in the preparation substances such as heparins or heparinoids, known for their anticoagulant properties, in order to obtain a synergic effect with carbon dioxide which helps dissolve blood clots.

The preparation of the invention can be used to prepare evacuation and cleansing enemas before surgery and endoscopic procedures, or in patients suffering from intestinal diverticuli, stenosis or atony, ulcerous colitis or Crohn's disease, to alleviate the symptoms, eliminate bacteria and exudative and excrementitious concretions, and facilitate penetration of active constituents and lymph absorption.

The preparation of the invention can be used to obtain anaesthetic effects if the gas dissolved in the water is an anaesthetic gas such as nitrous oxide.

The preparation of the invention can be administered with a rubber syringe or a squeeze bottle, optionally fitted with a spray device or a metering valve, or a portable variable preparation apparatus.

In practice, the composition of the preparation of the invention can differ from that indicated, while still remaining within the legal protection of the invention.

1. Preparation for douches and/or irrigations of natural or pathological cavities in the human body, characterised in that it comprises water to which at least one gaseous substance is added.

2. Preparation as claimed in claim 1, in which said at least one gaseous substance includes, either individually or in combination with one another carbon dioxide, oxygen, nitrogen, a gaseous anaesthetic substance or an inert gas.

3. Preparation as claimed in claim 2, in which said gaseous anaesthetic substance is nitrous oxide.

4. Preparation as claimed in any of the preceding claims, in which said water is still mineral water.

5. Preparation as claimed in any of claims 2 to 4, in which said carbon dioxide is dissolved in said water in a percentage ranging between approximately 1% and approximately 30%.

6. Preparation as claimed in claim 5, in which said carbon dioxide is dissolved in said water in a percentage of approximately 10%.

7. Preparation as claimed in one of claims 2 to 6, in which said oxygen is dissolved in said water in a quantity ranging between approximately 300 and approximately 600 mg/litre.

8. Preparation as claimed in any of the preceding claims, characterised in that it also includes one or more substances selected from a group comprising cleansing, deodorising, desensitising, anti-itching, anti-inflammatory, wound-healing, revulsive, eudermic and reparatory, antibacterial, antiparasitic, anti fungal, antiviral, antiexudative, corticosteroid, antitumoral, immunomodulating, haemostatic, haemorheological and hormonal substances.

9. Preparation as claimed in any of claims 1 to 7, which also includes active constituents to make a parenteral perfusion solution, which can also be used as a dialysis solution, for the peritoneal, pleural, pericardial or cranial joint cavity.

10. Preparation as claimed in any of claims 1 to 7, which also includes formed elements of blood or plasma.

11. Preparation as claimed in any of claims 1 to 7, or claim 8, which also includes heparins or heparinoids.

12. Preparation as claimed in any of claims 1 to 7, which also includes viral or genetic material as a carrier of DNA.

13. Preparation as claimed in claim 12, which also includes active constituents able to facilitate the passage of said viral material through the cell membranes.
